(19) United States
(12) Patent Application Publication
(10) Pub. No.: US 2005/0220853 A1
(43) Pub. Date: Oct. 6, 2005

(54) Controlled delivery of therapeutic agents from medical articles

(76) Inventors: Kinh-Luan (Lenny) Dao, Randolph, MA (US); Anastasia Panos, Worcester, MA (US); Yem Chin, Burlington, MA (US); Sheng-Ping (Samuel) Zhong, Shrewsbury, MA (US)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/816,677

(22) Filed: Apr. 2, 2004

(51) Int. Cl. A61K 9/70

(52) U.S. Cl. 424/449

(57) ABSTRACT

The present invention provides novel adenosine receptor antagonists, more particularly, A₁ adenosine receptor antagonists. Pharmaceutical compositions comprising an A₁ adenosine receptor antagonist disclosed herein and a pharmaceutically acceptable carrier are further provided. Compositions also include diagnostic assay-type probes comprising a novel A₁ adenosine receptor antagonist that is labeled or conjugated with radioactive or non-radioactive material. Methods for treating A₁ adenosine receptor related disorders comprising administering an A₁ adenosine receptor antagonist of the invention are also disclosed. The novel A₁ adenosine receptor antagonist compositions find further use in diagnostic and imaging methods.

### RELATED APPLICATION DATA

This application is related to U.S. Ser. No. 10/638,564 filed Aug. 11, 2003 and entitled “MEDICAL DEVICES COMPRISING SPRAY DRIED MICROPARTICLES,” which is incorporated by reference herein in its entirety.

### FIELD OF THE INVENTION

The present invention relates to medical articles that are useful for the controlled delivery of therapeutic agents, including high-molecular-weight therapeutic agents.

### BACKGROUND OF THE INVENTION

Percutaneous transluminal coronary angioplasty (“PTCA” or “angioplasty”) procedures have been performed for many years as an adjunct to correcting vascular disease in patients. Angioplasty procedures commonly involve the insertion of a catheter having a balloon through the patient's vascular system until the balloon is positioned across a lesion or blockage in a coronary artery. The balloon is then inflated to compress the lesion or blockage against the arterial walls, thereby opening the artery for increased blood flow.

In some instances, however, the goal of the angioplasty procedure is defeated at least in part by a complete or partial reclosure of the artery at or near the compressed lesion or blockage. Two mechanisms are believed to be principally responsible for reclosure of the artery. The first mechanism is recoil, which is a mechanical process involving the elastic rebound of the compressed lesion or blockage. The second mechanism is restenosis, which is believed to be caused by proliferation of the smooth muscle cells present in the artery walls near the lesion or blockage. Restenosis can occur over a period of several weeks or months after the PTCA procedure.

Many different methods have been employed to limit the effects of restenosis, including radiation treatments and various drug therapies, delivered locally and systemically, to slow proliferation of the smooth muscle cells. Recoil of the arterial walls can be prevented by using stents, which can be temporarily or permanently deployed within the artery to mechanically maintain patency of the artery. Stents are very effective at carrying out this task; however, they may also irritate the contacting arterial walls, thereby encouraging restenosis.

Gene therapy has been used for diverse medical purposes, including slowing the proliferation of smooth muscle cells. Genes are usually delivered into a patient's cells through a vector, for example, a retroviral vector whose DNA is genetically engineered to include a desired DNA sequence. Alternatively, nonviral gene transfer methods can be used, for example, plasmid DNA vectors (which can be delivered, for example, along with polymeric carriers, DNA condensing agents, lipofection agents, receptor mediated delivery vectors, and so forth).

Hence, incorporation of DNA molecules into the coronary artery walls near the treatment site in connection with angioplasty can be beneficial to inhibit restenosis. Moreover, a stent can be used as the delivery vehicle for the DNA, while at the same time maintaining the patency of the artery following PTCA.

One way to control the release of therapeutic agents, including DNA or other high-molecular-weight therapeutic agents, from stents or other medical articles is to first precipitate/deposit the therapeutic agent onto the surface of the medical article, and to subsequently provide a polymeric barrier layer over the therapeutic agent. This method, however, can be limited by a low affinity of the surface of the medical article for the therapeutic agent, and vice versa. For example, medical devices surfaces, including a wide range of metallic and polymeric surfaces, are commonly hydrophobic. Various therapeutic agents, including polynucleotides such as DNA and RNA, on the other hand, are relatively hydrophilic, leading to limited surface coverage and to loss of the therapeutic agent.

In addition, certain polymers that are highly biocompatible (e.g., polystyrene-polyisobutylene copolymers) may in some instances provide insufficient mass transport of therapeutic agents, particularly high-molecular-weight therapeutic agents such as polynucleotides, after deployment, thereby limiting the efficiency and control of the therapeutic-agent release.

Accordingly, there is a need for coatings for stents and other medical articles which release therapeutic agents, including high-molecular-weight therapeutic agents such as polynucleotides, in a controlled fashion, and which do not suffer from the foregoing and other disadvantages.

### SUMMARY OF THE INVENTION

The above and other challenges are met by the present invention. According to one aspect of the present invention, medical articles are provided which comprise the following: (a) an adhesive region comprising an adhesive, (b) a therapeutic agent and (c) optional microparticles. In this aspect of the invention, the therapeutic agent, the optional microparticles, or both the therapeutic agent and the optional microparticles are adhered to the adhesive region.

In certain embodiments, the medical article further comprises a substrate, for example, a polymeric, ceramic or metallic substrate, with the adhesive region disposed in a layer over at least a portion of the medical article substrate.

Upon placement of such medical articles at a position on or within a patient, therapeutic agent is released from the medical article and into the patient. Examples of medical articles include, for instance, implantable or insertable medical devices.

Other aspects of the present invention are directed to methods of providing such medical articles.

An advantage of the present invention is that medical articles can be provided, which regulate the release of therapeutic agents, including polynucelotides and other high-molecular-weight therapeutic agents, from a medical article to a patient.

Another advantage of the present invention is that medical articles can be provided which exhibit increased adherence between the therapeutic agents and polymeric and non-polymeric substrate surfaces of the medical articles, thereby increasing coating efficiency.

Another advantage of the present invention is that existing medical devices can be readily modified to provide them with therapeutic-agent releasing coatings.

These and other aspects, embodiments and advantages of the present invention will become immediately apparent to those of ordinary skill in the art upon review of the Detailed Description and claims to follow.

### DETAILED DESCRIPTION OF THE INVENTION

According to one aspect of the present invention, medical articles are provided which contain the following: (a) one or more adhesive regions, each containing one or more adhesives, (b) one or more therapeutic agents, and (c) optional microparticles. In this aspect of the invention, the therapeutic agent, the optional microparticles, or both the therapeutic agent and the optional microparticles are adhered to the adhesive region.

“Adhesives,” as the term is used herein, are materials that are capable of binding therapeutic agents and microparticles upon contact with the same. The adhesives utilized in connection with the present invention include the following: (1) Adhesives that are inherently capable of adhesion upon contact with therapeutic agents and with the optional microparticles (referred to herein as “pressure-sensitive adhesives”) (an everyday example of a pressure sensitive adhesive is the adhesive that is provided on an adhesive tape). (2) Adhesives that adhere upon contact with therapeutic agents and with the optional microparticles due to the presence of residual solvent, and that maintain adhesion subsequent to solvent removal (referred to herein as “solvent assisted adhesives”). (3) Adhesives that adhere upon contact with therapeutic agents and with the optional microparticles (due to the tackiness of the uncured adhesive), and which display increased adhesion after the adhesive undergoes a curing process (referred to herein as “curable adhesives”). In the latter case, the physical properties of the adhesive change as a result of chemical reactions that occur during curing (e.g., crosslihking and/or other reactions, based, for instance, on condensation, addition, substitution, and/or other reaction mechanisms). Chemical reaction within curable adhesives can be brought about in a number of ways known in the adhesive art, including the application of energy (e.g., heat, ultraviolet radiation or other radiation), the presence of an external chemical reactant or co-reactant (e.g., moisture-induced polymerization in the case of cyanoacrylates, and co-reactant-based polymerization in the case of two-component urethanes and two-component epoxies, among others), the removal of a solvent (e.g., upon drying), and so forth. The adhesives for use in the present invention include those that maintain their adhesive properties, whether wet or dry.

Suitable curable adhesives for use in connection with the present invention include synthetic and natural adhesives, which can be selected from one or more of the following among others: cyanoacrylates, epoxy-based adhesives, urethane-based adhesives, mussel adhesive proteins, fibrin glues, thrombin-based adhesives, silk-based adhesives, elastin-based adhesives, collagen-based adhesives, casein-based adhesives, gelatin-based adhesives, albumin-based adhesives, keratin-based adhesives, chitin-based adhesives, and chitosan-based adhesives, including natural proteins in combination with aldehyde or other crosslinkers (e.g., albumin-glutaraldehyde adhesives, gelatin-resorcinol-formaldehyde adhesives, and so forth).

In certain embodiments protein-based adhesives are utilized. A particularly desirable curable adhesive is mussel protein adhesive. The foot of the common mussel (Mytilus edulis) produces an adhesive that keeps the shelled organism anchored to rocks and other objects, allowing them to withstand the extreme pounding of waves. Chemical analysis of this natural, under-water-curable, water-proof glue has shown that the key to its adhesive properties is a unique compound called mussel adhesive protein, which contains a high concentration of an amino acid, DOPA (dihydroxyphenylalanine). Once secreted, mussel adhesive protein undergoes an in-situ crosslinking or hardening reaction, which leads to the formation of a solid adhesive that clings to wet surfaces with extraordinary strength.

Suitable pressure-sensitive adhesives for use in connection with the present invention include synthetic and natural adhesives, which can be selected from one or more of the following: hydrocolloid-based adhesives, acrylic based adhesives (e.g., 0175 Adhesive, Spectrum™ and Spectrum Plus™ from Velcro USA, Inc.), rubber-based adhesives, including natural rubber, polyisoprene, polyisobutylene, butyl rubber, acrylonitrile rubber, etc. (e.g., 19 Adhesive, Tempo™ and Vector™ from Velcro USA, Inc.), styrene-block-copolymer-based pressure-sensitive adhesives such as styrene-isoprene-styrene and styrene-butadiene-styrene block copolymer adhesives, silicone-based pressure-sensitive adhesives, and polyurethane-based pressure-sensitive adhesives, among others.

Suitable solvent-assisted adhesives for use in connection with the present invention include a wide range of synthetic and natural polymers, and can be selected from the polymeric materials listed below for use in microparticles.

In some embodiments of the invention, the adhesive region is in the form of an adhesive layer that covers all or a part of an underlying medical article substrate (e.g., where a metal substrate, such as a stent, or a polymeric substrate, such as a balloon or a patch, is coated with an adhesive layer in accordance with the present invention). As used herein a “layer” of a given material is a region of that material whose thickness is small compared to both its length and width. As used herein, a layer need not be planar, for example, taking on the contours of an underlying substrate. Layers can be discontinuous (e.g., patterned). Terms such as “film,” “layer” and “coating” may be used interchangeably herein. In other embodiments of the invention, the adhesive region corresponds to a component of a medical device. In still other embodiments, the adhesive region corresponds to the bulk of a medical article (e.g., where the adhesive region is cast in the form of a medical article using a mold or other template).

Methods by which an adhesive layer may be provided on an underlying substrate (e.g., a medical article substrate or a releasable substrate such as a mold or other template) are varied and include the following methods, among others: spraying techniques, roll and brush coating techniques, dipping techniques, spin coating techniques, web coating techniques, techniques involving coating via mechanical suspension such as air suspension, ink jet techniques, and electrostatic techniques. In some embodiments, layers are repeatedly applied to build up the thickness of the adhesive region.

In some embodiments of the invention, the adhesive region is a biodisintegrable adhesive region. As a result, the release of the therapeutic agent and/or optional microparticles is modulated based, at least in part, upon the disintegration rate of the adhesive region. This is advantageous, for example, because the therapeutic agent and/or microparticles are securely bonded to the medical article during delivery; at the same time, however, the rate at which the therapeutic agent and/or microparticles are released from the medical article is controlled. As used herein, a “biodisintegrable” adhesive is an adhesive which undergoes dissolution, degradation, resorption and/or other disintegration processes upon administration to a patient.

As noted above, in the medical articles of the present invention, therapeutic agent, the microparticles, or both the therapeutic agent and microparticles are adhered to the adhesive region. Hence, variations of the present invention include the following, among others: (a) the therapeutic agent is adhered to the adhesive region in the absence of the microparticles; (b) the medical article includes both therapeutic agent and microparticles, and the therapeutic agent is at least partially embedded or otherwise encapsulated within or attached to the microparticles, which are in turn adhered to the adhesive region; and (c) the medical article includes both therapeutic agent and microparticles, and the therapeutic agent is not at least partially embedded or otherwise encapsulated within or attached to the microparticles, in which case (i) the therapeutic agent and microparticles are both adhered to the adhesive region or (ii) the microparticles are adhered to the adhesive region and the therapeutic agent occupies interstices between the microparticles.

In some embodiments, the therapeutic agent and/or microparticles are applied in powder form to the surface of the adhesive region. Examples include: (a) applying the therapeutic agent and/or microparticles in powder form to a pressure-sensitive adhesive region, (b) applying the therapeutic agent and/or microparticles in powder form to the surface of a solvent-assisted adhesive that contains residual solvent, followed by solvent removal, and (c) applying the therapeutic agent and/or microparticles in powder form to an uncured adhesive region, followed by curing. Examples of techniques by which therapeutic agent and/or microparticles may be applied in powder form include, for example, spraying techniques, fluidized coating techniques, rapid prototyping head techniques, techniques involving rolling/dipping into powder, and so forth.

In some embodiments, the therapeutic agent and/or microparticles are dissolved or dispersed within a fluid (e.g., water and/or an organic solvent) which is applied to the adhesive region (e.g., by spraying techniques, roll and brush coating techniques, dipping techniques, spin coating techniques, web coating techniques, techniques involving coating via mechanical suspension such as air suspension, ink jet techniques, electrostatic techniques, etc.).

In some embodiments, the therapeutic agent and/or microparticles are applied to the adhesive region using a combination of the above techniques (e.g., applying the microparticles in powder form to the adhesive region, followed by application of a solution or dispersion of the therapeutic agent).

Steps of (a) applying an adhesive region, followed by (b) applying therapeutic agent and/or microparticles can be repeated as desired, thereby creating alternating regions of adhesive and therapeutic agent/microparticles. In these embodiments, the adhesive region is beneficially a biodisintegrable adhesive region, thereby promoting release of the therapeutic agent over time.

Numerous variations are possible in embodiments of the invention where the therapeutic agent is provided in conjunction with microparticles that are adhered to the adhesive region. For example, microparticles and therapeutic agent can be applied to the surface of the adhesive region, either sequentially or concurrently.

Where the microparticles and the therapeutic agent are applied sequentially, examples include the following: (A) Application of the therapeutic agent, followed by application of the microparticles, followed by solvent removal (in the case of a solvent assisted adhesive) or cure (in the case of a curable adhesive). Where a pressure sensitive adhesive is employed, no such final step is needed. (B) Application of the microparticles, followed by application of the therapeutic agent, followed by solvent removal (in the case of a solvent assisted adhesive) or cure (in the case of a curable adhesive). Again, where a pressure sensitive adhesive is employed, no such final step is needed. (C) Application of the microparticles, followed by solvent removal (in the case of a solvent assisted adhesive) or cure (in the case of a curable adhesive), followed by application of the therapeutic agent to the attached microparticle layer.

Where the microparticles and the therapeutic agent are provided concurrently, they are either provided as separate entities (e.g., where the microspheres create adjacent pockets which are occupied by the therapeutic agent and from which the therapeutic agent is released), or they are provided as a combined entity (e.g., where the therapeutic agent is at least partially embedded or otherwise encapsulated within or attached to the microparticles). In embodiments wherein the therapeutic agent is at least partially embedded or otherwise encapsulated within or attached to the microparticles (e.g., where the microparticles provide a drug delivery matrix within which the therapeutic agent is dispersed, or where the microparticles include an inner region comprising the therapeutic agent which is encapsulated by another material, or where the therapeutic agent is attached to the surface of the microparticle, etc.), the therapeutic agent is commonly released from the microparticles by diffusing from the microparticles, by biodisintegration of the microparticles, by cleavage of a bond between the microparticles and the therapeutic agent, and so forth.

Microparticles for use in connection with the present invention are available in a wide range of shapes, sizes, and compositions. Microparticles of different sizes and/or of different shapes and/or of different compositions can be admixed within a single microparticle-containing region, if desired. Moreover, in embodiments where two or more distinct microparticle-containing regions are provided (e.g., separate microparticle-containing regions lying over one another or separate microparticle-containing regions lying adjacent to one another) the regions can comprise microparticles of different sizes and/or of different shapes and/or of different compositions.

Microparticle shapes include spherical, rod shaped, irregular, and so forth. Microparticles can be solid or hollow.

Average (e.g., weight average) microparticle size typically ranges from 10 nm to 1000 μm, more typically from 0.1 to 50 μm, in largest linear cross-sectional dimension (i.e., the diameter in the case of microspheres, the length in the case of filamentous microparticles, etc.). In many embodiments, the release rate of the therapeutic agent depends upon the size of the microparticles. For example, bigger particles create bigger pockets for occupation by the therapeutic agent and therefore can modulate release. As another example, where the microparticles provide a drug delivery matrix within which the therapeutic agent is dispersed, bigger particles present longer paths for diffusion and/or take longer to disintegrate, again modulating release.

In certain embodiments, the microparticles utilized either inherently have a surface charge, or they are provided with one. The presence of a surface charge on the microparticles is frequently advantageous, for example, because the particles resist agglomeration, thereby promoting the formation of more evenly distributed pockets at the surface of the medical articles of the present invention which the therapeutic agent can occupy. Moreover, the presence of a surface charge on the microparticles can also be used to attract charged or polar therapeutic agents in certain embodiments, thereby increasing drug loading.

As to composition, the microparticles can comprise ceramic, metallic and polymeric materials, including microparticles that are biodisintegrable and microparticles that are not (sometimes referred to herein as biostable).

Examples of ceramic materials for use in the microparticles of the present invention can be selected from those comprising one or more of the following: metal oxides, including aluminum oxides and transition metal oxides (e.g., oxides of titanium, zirconium, hafnium, tantalum, molybdenum, tungsten, rhenium, and iridium); silicon-based ceramics, such as those containing silicon nitrides, silicon carbides and silicon oxides (sometimes referred to as glass ceramics); calcium phosphate ceramics (e.g., hydroxyapatite); and carbon-based ceramic-like materials such as carbon nitrides.

Examples of metallic materials for use in the microparticles of the present invention can be selected from those comprising one or more of the following: metal alloys such as cobalt-chromium alloys, nickel-titanium alloys (e.g., nitinol), cobalt-chromium-iron alloys (e.g., elgiloy alloys), nickel-chromium alloys (e.g., inconel alloys), and iron-chromium alloys (e.g., stainless steels, which contain at least 50% iron and at least 11.5% chromium), and noble metals such as silver, gold, platinum, palladium, iridium, osmium, rhodium, titanium, tungsten, and ruthenium.

Examples of polymeric materials for use in the microparticles of the present invention can be selected from those comprising one or more of the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydoxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, polystyrenes, styrene-maleic anhydride copolymers, styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; polyalkyl oxide polymers and copolymers including polyethylene oxides (PEO); glycosaminoglycans; polyesters including polyethylene terephthalates and aliphatic polyesters such as polymers and copolymers of lactide (which includes lactic acid as well as d-,l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyolefin polymers and copolymers, including polyalkylenes such as polypropylenes, polyethylenes (low and high density, low and high molecular weight), polybutylenes (such as polybut-1-ene and polyisobutylene), EPDM copolymers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as blends and copolymers of the above.

Such polymers may be provided in a variety of configurations, including cyclic, linear and branched configurations. Branched configurations include star-shaped configurations (e.g., configurations in which three or more chains emanate from a single branch point), comb configurations (e.g., graft polymers having a main chain and a plurality of branching side chains), and dendritic configurations (e.g., arborescent and hyperbranched polymers). The polymers can be formed from a single monomer (i.e., they can be homopolymers), or they can be formed from multiple monomers (i.e., they can be copolymers) that can be distributed, for example, randomly, in an orderly fashion (e.g., in an alternating fashion), or in blocks.

In certain beneficial embodiments, the polymeric materials for use in the microparticles of the present invention can be selected from biocompatible biodisintegrable protein materials, which may be, for example, synthetic proteins, genetically-engineered proteins, natural proteins or any combination thereof. Naturally occurring proteins that may be utilized are selected, for example, from elastin, collagen, albumin, keratin, fibronectin, silk, silk fibroin, silk elastin, actin, myosin, fibrinogen, thrombin, aprotinin, antithrombin III, and any other biocompatible, biodisintegrable protein. Further specific biocompatible, biodisintegrable protein materials are described, for example, in U.S. Patent Appln. No. 2003/0007991.

Using stimuli sensitive protein based microparticles allows for a high degree of control of the material making up the microparticles and, thus, the release profile of therapeutic agents from the microparticles. For example, in one embodiment of the invention, protein-based silk-elastin microparticles with associated DNA (e.g., microparticles with DNA that is entrapped, encapsulated, adsorbed and/or absorbed) are adhered to an adhesive region. Direct mixing of the silk-elastin microparticles with the DNA in aqueous solution results in the association of the DNA (as well as other drugs) with the microparticles. In general, the higher the number of silk units in such microparticles, the higher the degree of crosslinking that exists within the microparticles, and thus the lower the degradation rate. The pore sizes of such particles are typically on the order of 0.1 μm, however, pore size and swelling can be controlled by varying the number of silk units, the pH, the temperature, and so forth.

Medical articles which can be provided in accordance with the present invention include essentially any medical article from which release of a therapeutic agent is desired. Examples of medical articles include patches for delivery of therapeutic agent to intact skin, broken skin (including wounds), and surgical sites. Examples of medical articles also include implantable or insertable medical devices, for example, catheters (for example, renal or vascular catheters such as balloon catheters), guide wires, balloons, filters (e.g., vena cava filters), stents (including coronary vascular stents, cerebral, urethral, ureteral, bone prosthesis, biliary, tracheal, gastrointestinal and esophageal stents), stent grafts, cerebral aneurysm filler coils (including Guglilmi detachable coils and metal coils), vascular grafts, myocardial plugs, patches (e.g., vascular patches such as heart cavity patches, gastrointestinal patches such as esophageal or stomach patches, and urological patches such as bladder, kidney, ureteral and urethral patches), pacemakers and pacemaker leads, heart valves, as well as any other medical device that is implanted or inserted into the body and from which a therapeutic agent is released.

The medical articles of the present invention include medical articles that are used for either systemic treatment or for the localized treatment of any mammalian tissue or organ. Non-limiting examples are tumors; organs including the heart, coronary and peripheral vascular system (referred to overall as “the vasculature”), lungs, trachea, esophagus, brain, liver, kidney, bladder, urethra and ureters, eye, intestines, stomach, pancreas, ovary, and prostate; skeletal muscle; smooth muscle; breast; dermal tissue; cartilage; and bone. As used herein, “treatment” refers to the prevention of a disease or condition, the reduction or elimination of symptoms associated with a disease or condition, or the substantial or complete elimination a disease or condition. Preferred subjects are mammalian subjects and more preferably human subjects.

For example, in accordance with one specific embodiment of the invention, the medical device is a patch for the application of gene therapy to heart cavities. In this particular embodiment, microspheres are adhered to one side of the patch with mussel protein adhesive, and plasmid DNA is disposed within the pockets created by the microspheres after cure. On the opposite side of the patch, the same adhesive can be used to anchor a repellant species, for example, polyethylene glycol. The patch can be delivered, for example, using a balloon.

In accordance with another specific embodiment of the invention, a balloon is utilized as a substrate for an adhesive coating. A therapeutic agent, for example a high molecular weight therapeutic agent (e.g., plasmid DNA) and microparticles are then applied to the adhesive coating while in a sticky state. The balloon is subsequently used for PCTA, while at the same time providing a therapeutic agent to the site of the lesion or blockage. In accordance with yet another specific embodiment of the invention, a therapeutic agent, for example a high-molecular-weight therapeutic agent (e.g., plasmid DNA), is contacted with a biodisintegrable adhesive region while in a sticky state, and the resulting composition is exposed to the body via balloon or stent insertion. Where the therapeutic agent is adhered to the adhesive region, the adhesive region is typically biodisintegrable. Where the therapeutic agent is not adhered to the adhesive region, the adhesive region is typically either biodisintegrable or biostable.

In some embodiments of the invention, an implantable or insertable medical device is further provided with an optional biodisintegrable protection layer to prevent premature loss of the therapeutic agent. The biodisintegrable protection layer covers and protects the therapeutic-containing regions of the device during deployment, but disintegrates (e.g., dissolves or is enzymatically or hydrolytically degraded) after being situated at a location within a patient, thereby allowing the therapeutic agent to be released.

The present invention is especially useful in delivering high-molecular-weight therapeutic agents, which are defined herein to include therapeutic agents having a molecular weight greater than 500, typically greater than 1,000, more typically greater than 2,000, or combinations of agents which contain at least one therapeutic agent having such molecular weights (and which can also contain non-high-molecular-weight therapeutic agents, referred to herein as “low-molecular-weight therapeutic agents”). “Therapeutic agents,” “pharmaceutically active agents,” “pharmaceutically active materials,” “drugs” and other related terms may be used interchangeably herein and include genetic therapeutic agents, non-genetic therapeutic agents and cells. High and low-molecular weight therapeutic agent can be selected from suitable members of the lists of therapeutic agents to follow.

Exemplary non-genetic therapeutic agents for use in connection with the present invention include: (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) anti-neoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (O) agents that interfere with endogenous vasoactive mechanisms; (p) inhibitors of leukocyte recruitment, such as monoclonal antibodies; (q) cytokines and (r) hormones.

Some exemplary non-genetic therapeutic agents include paclitaxel, sirolimus, everolimus, tacrolimus, cladribine, dexamethasone, estradiol, ABT-578 (Abbott Laboratories), trapidil, liprostin, Actinomcin D, Resten-NG, Ap-17, abciximab, clopidogrel and Ridogrel.

Exemplary genetic therapeutic agents for use in connection with the present invention include anti-sense DNA and RNA as well as DNA coding for: (a) anti-sense RNA, (b) tRNA or rRNA to replace defective or deficient endogenous molecules, (c) angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine, polyethyleneimine (PEI)), graft copolymers (e.g., polyether-PEI and polyethylene oxide-PEI), neutral polymers PVP, SP1017 (SUPRATEK), lipids such as cationic lipids, liposomes, lipoplexes, nanoparticles, or microparticles, with and without targeting sequences such as the protein transduction domain (PTD).

Cells for use in connection with the present invention include cells of human origin (autologous or allogeneic), including whole bone marrow, bone marrow derived mono-nuclear cells, progenitor cells (e.g., endothelial progenitor cells), stem cells (e.g., mesenchymal, hematopoietic, neuronal), pluripotent stem cells, fibroblasts, myoblasts, satellite cells, pericytes, cardiomyocytes, skeletal myocytes or macrophage, or from an animal, bacterial or fungal source (xenogeneic), which can be genetically engineered, if desired, to deliver proteins of interest.

Numerous therapeutic agents, not necessarily exclusive of those listed above, have been identified as candidates for vascular treatment regimens, for example, as agents targeting restenosis. Such agents include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) ACE inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (1) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (O) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, fluvastatin, simvastatin and cerivastatin, (u) fish oils and omega-3-fatty acids, (v) free-radical scavengers/antioxidants such as probucol, vitamins C and E, ebselen, trans-retinoic acid and SOD mimics, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) MMP pathway inhibitors such as marimastat, ilomastat and metastat, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, paclitaxel and epothilone), caspase activators, proteasome inhibitors, angiogenesis inhibitors (e.g., endostatin, angiostatin and squalamine), rapamycin, cerivastatin, flavopiridol and suramin, (aa) matrix deposition/organization pathway inhibitors such as halofuginone or other quinazolinone derivatives and tranilast, (bb) endothelialization facilitators such as VEGF and RGD peptide, and (cc) blood rheology modulators such as pentoxifylline.

Numerous additional therapeutic agents are also disclosed in U.S. Pat. No. 5,733,925 assigned to NeoRx Corporation, the entire disclosure of which is incorporated by reference.

As noted above, the present invention is especially useful in delivering high-molecular-weight therapeutic agents. Examples of high-molecular-weight therapeutic agents, not necessarily exclusive of those listed above, include polysaccharide therapeutic agents having a molecular weight greater than 1,000; polypeptide therapeutic agents having a molecular weight greater than 10,000; polynucleotides, including antisense polynucleotides, having a molecular weight greater than 2,000, gene-encoding polynucleotides, including plasmids, having a molecular weight greater than 500,000; viral and non-viral particles having a diameter greater than about 50 nanometers, and cells.

A “polynucleotide” is a nucleic acid polymer. A polynucleotide can include both double- and single-stranded sequences, and can include naturally derived and synthetic DNA sequences. The term also includes sequences that include any of the known base analogs of DNA and RNA, and includes modifications, such as deletions, additions and substitutions (generally conservative in nature) to native sequences.

Typical polynucleotide therapeutic agents include the genetic therapeutic agents specifically listed above, and more generally include DNA encoding for various polypeptide and protein products including those previously listed. Some additional examples of polynucleotide therapeutic agents include DNA encoding for the following: cytokines such as colony stimulating factors (e.g., granulocyte-macrophage colony-stimulating factor), tumor necrosis factors (e.g., fas ligand) and interleukins (e.g., IL-10, an anti-inflammatory interleukin), as well as protease inhibitors, particularly serine protease inhibitors (e.g., SERP-1), tissue inhibiting metalloproteinases (e.g., TIMP-1, TIMP-2, TIMP-3, TIMP-4), monocyte chemoattractant proteins (e.g., MCP-1), protein kinase inhibitors including cyclin-dependent kinase inhibitors (e.g., p27, p21), endogenous and inducible nitric oxide synthase, CO-generating enzymes, such as hemoxygenases, which catalyze the oxidation of heme into the biologically active molecules iron biliverdin and CO (e.g., HOI-1), antiproliferative compounds, such as HKIS in a transdominant mutant peptide form, which are capable of interfering with the ability of endogenous HKIS to phosphorylate p27 thereby enhancing cell cycle arrest, as well as derivatives of the foregoing.

The term “polypeptide” refers to a polymer of amino acid residues. Both full-length proteins and fragments thereof are encompassed by the definition. The terms also include modifications, such as deletions, additions and substitutions (generally conservative in nature), to native sequence. Exemplary polypeptides include any of the polypeptides/proteins listed in the preceding paragraphs.

The term “polysaccharide” refers to a polymer of monosaccharide residues. Examples of polysaccharides include the polysaccharides listed in the preceding paragraphs. Low and high molecular weight heparin and dextran, including derivatives of the same, for example, dextran sulfate salts and dextran-metal complexes such as dextran-iron complex, are some exemplary polysaccharides.

Hybrids of the above high-molecular-weight therapeutics (e.g., DNA/protein hybrids and polysaccharide/protein hybrids) are also within the scope of the present invention.

Some specific classes of therapeutic agents are anti-proliferative agents, anti-inflammatory agents, anti-thrombotic agents, lipid mediators, vasodilators, anti-spasm agents, remodeling agents, endothelial-cell specific mitogens, as well as nucleotide sequences (which may further include an associated delivery vector) encoding for therapeutic agents having any one or combination of these therapeutic effects. Examples include plasmids that encode an antiproliferative protein within the arterial walls to help prevent a recurring blockage due to restenosis, anti-inflammatory proteins and anti-thrombotic polysaccharides designed to prevent blood clotting.

A wide range of therapeutic agent loadings can be used in connection with medical articles of the present invention, with the therapeutically effective amount being readily determined by those of ordinary skill in the art and ultimately depending, for example, upon the condition to be treated, the age, sex and condition of the patient, the nature of the therapeutic agent, the nature of the release region, the nature of the medical article, and so forth.

Although various embodiments are specifically illustrated and described herein, it will be appreciated that modifications and variations of the present invention are covered by the above teachings and are within the purview of the appended claims without departing from the spirit and intended scope of the invention.

1. A medical article comprising:
(a) an adhesive region comprising an adhesive; and
(b) a therapeutic agent, wherein said therapeutic agent is adhered to a surface of said adhesive region,
with the proviso that said therapeutic agent is adhered to said surface of said adhesive region in a form other than within spray dried microparticles that comprise said therapeutic agent and a carrier polymer.

2. The medical article of claim 1, wherein said adhesive is a curable adhesive.

3. The medical article of claim 2, wherein said adhesive is a curable, protein-based adhesive.

4. The medical article of claim 1, wherein said adhesive is a pressure-sensitive adhesive.

5. The medical article of claim 1, wherein said adhesive is a solvent assisted adhesive.

6. The medical article of claim 1, wherein said therapeutic agent is adhered to said adhesive region in the absence of said microparticles.

7. The medical article of claim 6, wherein said therapeutic agent is applied in powder form to said adhesive region.

8. The medical article of claim 6, wherein at least a portion of said adhesive region is biodisintegrable.

9. The medical article of claim 1, further comprising microparticles at least a portion of which are attached to said surface of said adhesive region.

10. The medical article of claim 9, wherein said microparticles are microspheres.

11. The medical article of claim 10, wherein said microspheres have a number average diameter between 0.1 to 50 μm.

12. The medical article of claim 9, wherein said medical article comprises at least two populations of microparticles that are of different composition from one another.

13. The medical article of claim 9, wherein said medical article comprises at least two populations of microparticles having different size distributions.

14. The medical article of claim 9, at least a portion of said therapeutic agent is attached to or is partially or fully encapsulated within said microparticles.

15. The medical article of claim 14, wherein said microparticles are biodisintegrable.

16. The medical article of claim 9, at least a portion of said therapeutic agent is neither attached to nor partially or fully encapsulated within said microparticles.

17. The medical article of claim 16, wherein said medical article is provided by a process in which said therapeutic agent is admixed in powder form with said microparticles and applied to said adhesive region.

18. The medical article of claim 16, wherein said medical article is provided by a process in which said microparticles are applied to said adhesive region in powder form, followed by the application of said therapeutic agent in powder form.

19. The medical article of claim 16, wherein said microparticles are biostable.

20. The medical article of claim 16, wherein said microparticles are biodisintegrable.

21. The medical article of claim 16, wherein at least a portion of said therapeutic agent is unadhered to said adhesive region and occupies interstices between said microparticles.

22. The medical article of claim 16, wherein at least a portion of said therapeutic agent is adhered to said adhesive region.

23. The medical article of claim 22, wherein said adhesive region is biodisintegrable.

24. The medical article of claim 1, wherein said medical article further comprises an additional biodisintegrable adhesive region over said therapeutic agent.

25. The medical article of claim 24, further comprising additional therapeutic agent adhered to a surface of said additional adhesive region.

26. The medical article of claim 1, further comprising a medical article substrate, wherein said adhesive region is disposed in a layer over at least a portion of said medical article substrate.

27. The medical article of claim 1, wherein said medical article comprises first and second distinct adhesive regions, which may be of the same or of different composition.

28. The medical article of claim 1, wherein said medical article comprises first and second therapeutic agents of different composition.

29. The medical article of claim 9, wherein said medical article comprises first and second populations of microparticles of different composition.

30. The medical article of claim 9, wherein said medical article comprises first and second populations of microparticles of different size distribution.

31. The medical article of claim 1, wherein said therapeutic agent is a high-molecular-weight therapeutic agent.

32. The medical article of claim 31, wherein said high-molecular-weight therapeutic agent is a polynucleotide-containing therapeutic agent.

33. The medical article of claim 1, wherein said adhesive is mussel protein adhesive.

34. The medical article of claim 9, wherein said medical article comprises:
(a) a first therapeutic agent and a first population of microparticles adhered to a surface of a first adhesive region; and
(b) a second therapeutic agent and a second population of microparticles adhered to a surface of said a second adhesive region which is distinct from said first adhesive region,
wherein said first and second adhesive regions may be of the same or different compositions,
wherein said first and second therapeutic agents may be of the same or different compositions, and
wherein said first and second populations of microparticles may be of the same or different composition or of the same or different size distribution.

35. The medical article of claim 34, wherein said first and second adhesive regions are of different composition.

36. The medical article of claim 34, wherein said first and second therapeutic agents are of different composition.

37. The medical article of claim 34, wherein said first and second populations of microparticles are of different composition.

38. The medical article of claim 34, wherein said first and second populations of microparticles are of different size distribution.

39. The medical article of claim 9, further comprising a biodisintegrable layer over said therapeutic agent and said microparticles.

40. The medical article of claim 1, wherein said medical article is an implantable or insertable medical device.

41. The medical article of claim 40, wherein said implantable or insertable medical device is selected from a balloon, a catheter, a stent, a patch, a heart valve, a bone prosthesis, a pacemaker, a vena cava filter, and an abnormal abdominal aneurism device.

42. The medical article of claim 40, wherein said implantable or insertable medical device further comprises a biodisintegrable protection layer disposed over said therapeutic agent to protect said therapeutic agent during device delivery.
